(19) United States
(12) Patent Application Publication
(10) Pub. No.: US 2026/0248568 A1
(43) Pub. Date: Aug. 27, 2026

(54) METHOD AND SYSTEM FOR PERFORMING BONE RESECTION IN ROBOTIZED COMPUTER-ASSISTED SURGERY

(71) Applicant: ORTHOSOFT ULC, Montreal (CA)

(72) Inventor: Pierre COUTURE, Montreal (CA)

(21) Appl. No.: 19/548,215

(22) Filed: Feb. 24, 2026

Related U.S. Application Data
(60) Provisional application No. 63/764,158, filed on Feb. 27, 2025.

(51) Int. Cl. A61B 34/30; A61B 17/15; A61B 17/16; A61B 34/10; A61B 34/20; B25J 9/16

(52) U.S. Cl. CPC A61B 34/30; A61B 17/155; A61B 17/1622; A61B 34/10; A61B 34/20; B25J 9/1671; A61B 2034/102; A61B 2034/105; A61B 2034/107

(57) ABSTRACT

A system for performing bone resection on a bone in robotized computer-assisted surgery, comprising: a processing unit; and a non-transitory computer-readable memory communicatively coupled to the processing unit and comprising computer-readable program instructions executable by the processing unit for: tracking a robot arm and a bone in a common referential system; obtaining a virtual model of a cutting block to be used on the bone, the virtual model including slots corresponding to at least two cutting planes of the cutting block; and controlling the robot arm having the cutting block supported at an end thereof to place the cutting block in a desired location on the bone, using the model of the cutting block and the tracking.

### CROSS-REFERENCE TO RELATED APPLICATION

The present application claims the benefit of United States Patent Application No. 63/764,158, filed on February 27, 2025, the entire contents of which are incorporated herein by reference.

### TECHNICAL FIELD

The present application relates to bone and tool tracking in computer-assisted orthopedic surgery and in robotized computer-assisted surgery, such as during resection of bone surfaces to receive implants.

### BACKGROUND OF THE ART

The navigation of surgical instruments or tools is an integral part of computer-assisted surgery (hereinafter “CAS”). The tools are navigated, i.e., tracked for position and/or orientation, in such a way that relative information pertaining to bodily parts is obtained. The information may be used in various interventions (e.g., orthopedic surgery, neurological surgery) with respect to the body, such as bone alterations, implant positioning, incisions and the like during surgery.

Moreover, robots are increasingly used in surgery. Robotized computer-assisted surgery typically feature a robot arm that supports various types of instruments. The stiffness of the robot arm, and the control of its movements as combined with navigation data generally contribute to the precision of a robotized computer-assisted procedure.

However, for freedom of movement, the robot arms are often serial robot arms in which links are interconnected by joints, with an end effector at a cantilevered free end of the robot arm. In spite of the stiffness characteristics of robot arms, and their precision of movements, in some instances loads associated with bone resection and other maneuvers may cause slight imprecisions in bone alterations.

### SUMMARY

In accordance with a first aspect of the present disclosure, there is provided a system for performing bone resection on a bone in robotized computer-assisted surgery, comprising: a processing unit; and a non-transitory computer-readable memory communicatively coupled to the processing unit and comprising computer-readable program instructions executable by the processing unit for: tracking a robot arm and a bone in a common referential system; obtaining a virtual model of a cutting block to be used on the bone, the virtual model including slots corresponding to at least one cutting plane of the cutting block; and controlling the robot arm having the cutting block supported at an end thereof to place the cutting block in a desired location on the bone, using the model of the cutting block and the tracking.

Further in accordance with the first aspect, for instance, the computer-readable program instructions are executable by the processing unit for recording points on the bone in the common referential system, using the robot arm.

Still further in accordance with the first aspect, for instance, the bone is a femur and wherein the computer-readable program instructions are executable by the processing unit for controlling the robot arm in the desired location against a distal cut of the femur.

Still further in accordance with the first aspect, for instance, the computer-readable program instructions are executable by the processing unit for obtaining the virtual model of the cutting block with the at least one cutting plane being an anterior cut plane and a posterior cut plane.

Still further in accordance with the first aspect, for instance, the computer-readable program instructions are executable by the processing unit for obtaining the virtual model of the cutting block with the cutting planes including an anterior chamfer cut plane and a posterior chamfer cut plane.

Still further in accordance with the first aspect, for instance, the computer-readable program instructions are executable by the processing unit for obtaining a virtual model of a distal cutting block to be used on the femur, the virtual model including at least a cutting plane corresponding to a distal cut of the femur.

Still further in accordance with the first aspect, for instance, the computer-readable program instructions are executable by the processing unit for controlling the robot arm having the distal cutting block supported at an end thereof to place the distal cutting block in a desired location on the femur, using the model of the distal cutting block and the tracking.

Still further in accordance with the first aspect, for instance, the computer-readable program instructions are executable by the processing unit for controlling the robot arm to perform a distal cut.

Still further in accordance with the first aspect, for instance, the computer-readable program instructions are executable by the processing unit for controlling the robot arm to drill a peg hole in the femur.

Still further in accordance with the first aspect, for instance, the computer-readable program instructions are executable by the processing unit for controlling the robot arm having a cutting tool supported at the end thereof to position itself relative to the cutting block.

Still further in accordance with the first aspect, for instance, the computer-readable program instructions are executable by the processing unit for obtaining the virtual model of the cutting block from a manufacturer file.

Still further in accordance with the first aspect, for instance, the computer-readable program instructions are executable by the processing unit for: tracking a tool relative to the bone and to the cutting block; merging virtual models of the tool and the bone to the tool and the bone in the tracking; calculating a location of a working end of the tool relative to the bone using the tracking, in a concealed condition of the working end of the tool relative to the bone while in the cutting block; and outputting the location of the working end of the tool relative to the bone.

Still further in accordance with the first aspect, for instance, the computer-readable program instructions are executable by the processing unit for indicating a proximity of the working end with a boundary of the bone.

Still further in accordance with the first aspect, for instance, the computer-readable program instructions are executable by the processing unit for stopping an actuation of the tool when the working end is at the boundary of the bone.

Still further in accordance with the first aspect, for instance, the computer-readable program instructions are executable by the processing unit for imaging and displaying the concealed working end of the tool relative to the bone.

Still further in accordance with the first aspect, for instance, the robot arm is part of the system.

Still further in accordance with the first aspect, for instance, the cutting block is part of the system.

Still further in accordance with the first aspect, for instance, the cutting block has at least four cut slots.

Still further in accordance with the first aspect, for instance, a saw is configured for performing planar cuts of the bone as used with the cutting block.

Still further in accordance with the first aspect, for instance, a drill is configured for drilling holes in the bone to secure the cutting block to the bone.

### DESCRIPTION OF THE DRAWINGS

FIG. 1 is a block diagram of a computer-assisted surgery (CAS) system with head-mounted navigation in accordance a variant with the present disclosure;

FIG. 2 is a perspective view and block diagram of a head-mounted device in accordance with another variant of the present disclosure;

FIG. 3A is a perspective view of a saw tool that may be used with the CAS system of FIG. 1;

FIG. 3B is a perspective view of a drilling tool that may be used with the CAS system of FIG. 1;

FIG. 4A is a perspective view of a femoral cutting block used with the CAS system of FIG. 1;

FIG. 4B is a view of a model of cut plane associated with the femoral cutting block of FIG. 1;

FIG. 5 is a flow chart showing a method for resecting a bone in robotized CAS, such as the CAS system of FIG. 1, in accordance with a variant of the present disclosure;

FIG. 6 is a perspective view of the CAS system of FIG. 1 during a placement of the femoral cutting block of FIG. 5 on the femur, with assistance from a robot arm;

FIG. 7A is a perspective view of the CAS system of FIG. 6, during resection using the femoral cutting block, with assistance of a robotic arm;

FIG. 7B is a perspective view of the CAS system of FIG. 6, during resection using the femoral cutting block, without assistance of a robotic arm; and

FIG. 8 is a perspective view of a saw tool that may be used with the CAS system of FIG. 1, with a patient specific guide.

### DETAILED DESCRIPTION

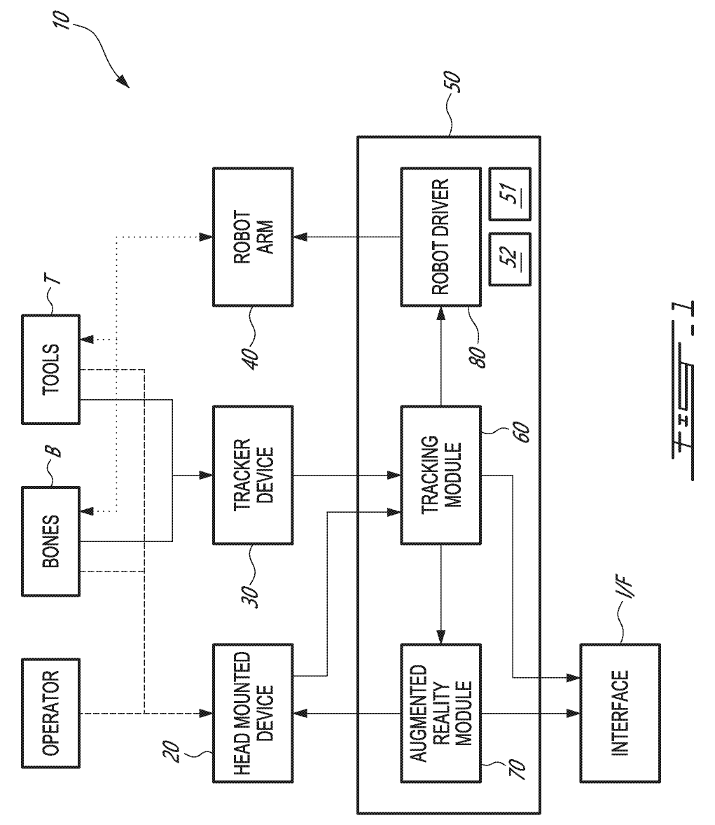

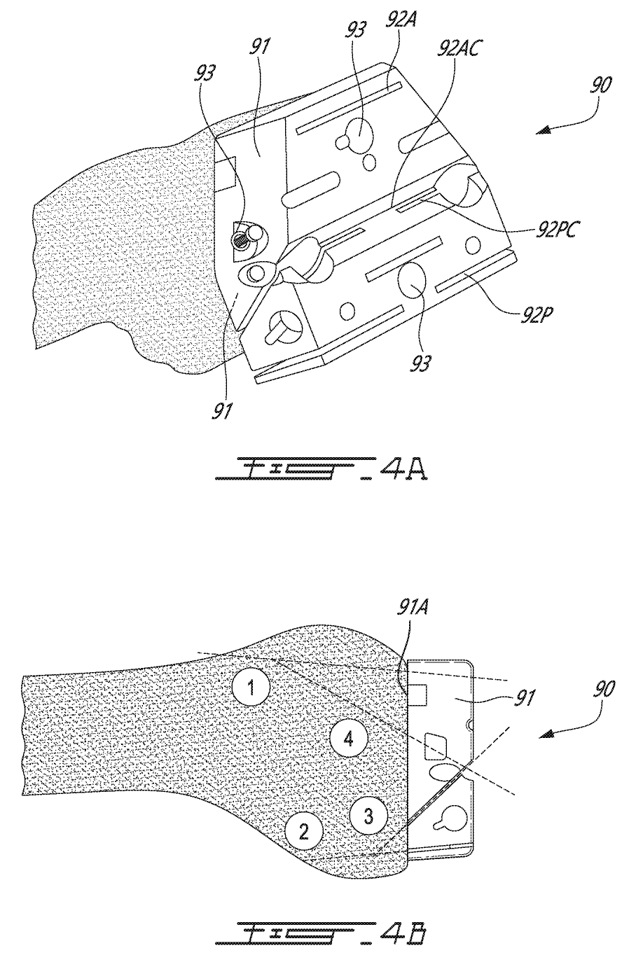

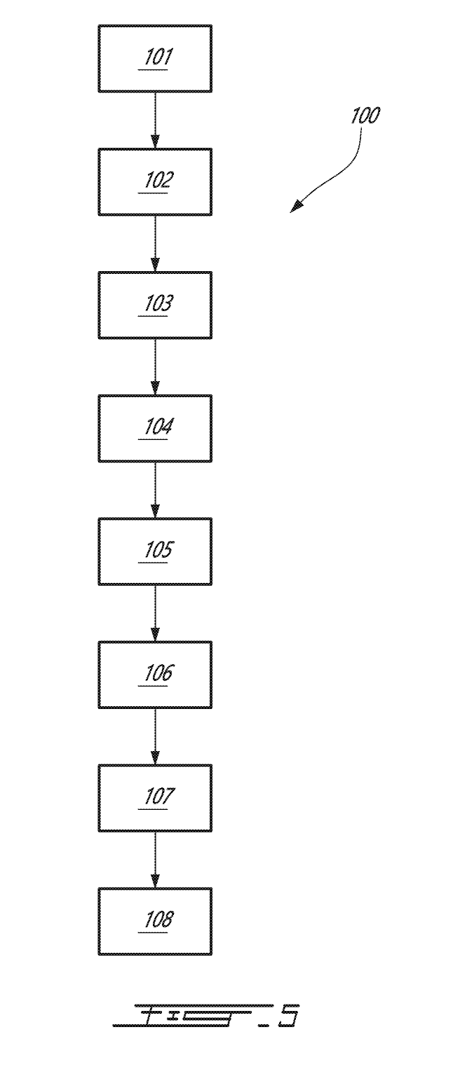

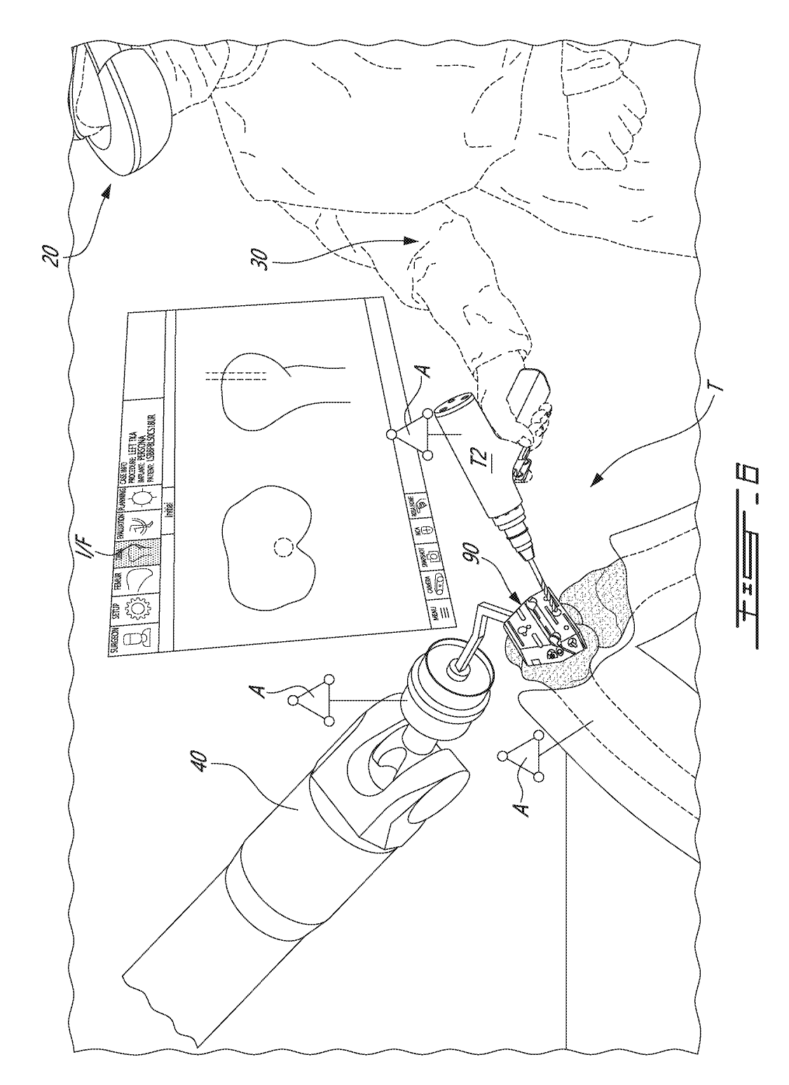

Referring to the drawings and more particularly to FIG. 1, computer-assisted surgery (CAS) system with optional head-mounted navigation is generally shown at 10, and is used to provide surgery assistance to an operator. For example, the CAS system 10 may be used to assist an operator wearing a head-mounted device with display in performing surgical maneuvers on a patient, such as surgical maneuvers associated with orthopedic surgery, including pre-operative analysis of range of motion, and implant assessment planning, as described hereinafter. In FIGS. 4 and 5, the system 10 is shown relative to a patient’s knee joint in supine decubitus, to perform the resection of the femur with a view of implanting a knee femoral implant component, also known as a distal femur implant, femoral component, etc. The system 10 could be used for other body parts, including non-exhaustively hip joint, spine, and shoulder bones, or in other types of surgery.

The CAS system 10 may be robotized in a variant, and has, may have or may be used with a head-mounted device 20, a tracker device 30, a robot arm 40, a CAS controller 50, a tracking module 60, an augmented reality module 70, a robot driver 80, and another tracking device 90, and/or any combination thereof:

The head-mounted device 20 is worn by an operator, such as by the surgeon performing surgery, and may be referred to as head-mounted tracking device 20 as it has the capacity to capture images, such as in video format. The head-mounted device 20 may have a display screen to provide data to the wearer, though this may be optional in an embodiment. For simplicity, the expressions head-mounted tracking device 20 and head-mounted device 20 are used interchangeably in the present disclosure and figures. The head-mounted tracking device 20 may be used to provide a display in augmented/mixed and/or virtual reality to a user. The head-mounted tracking device 20 may also be tasked with taking images of the surgery, with the images being used for the tracking of patient tissue (such as bones) and tools, for instance as a video feed. The head-mounted tracking device 20 may also be used as an interface by which an operator may communicate commands to the CAS system 10.

The tracking device 30, also known as a sensor device, apparatus, etc performs optical tracking of optical trackers A, so as to enable the tracking in space (a.k.a., navigation) of the robot 40, the patient and/or tools. The tracking device 30 may complement the tracking performed with the imaging done with the head-mounted tracking device 20, or may be the primary tracking feed of the CAS system 10. The tracking device 30 may employ camera technology similar to that of the head-mounted tracking device 20, such as depth cameras, with optional pattern projector, as described below, or may se a different imaging technology, to provide its video feed. The tracking device 30 may be said to be stationary. The tracking device 30 may be the primary tracking device, if the head-mounted tracking device 20 is absent or temporarily out of the line of the surgical scene, or with the head-mounted tracking device 20 providing complementary tracking capability. For example, the tracking device 30 may be a Navitracker® device, that used with trackable references A (a.k.a., trackers A), such as shown in FIG. 5. The tracking device 30 with trackers A may be used in any of the embodiments described herein. A Navitracker® tracks by triangulation trackable references A.

The robot arm 40 may optionally be present as the working end of the system 10 – the CAS system 10 could also be non robotic -, and may be used to guide or to perform bone alterations as planned by an operator and/or the CAS controller 50 and as controlled by the CAS controller 50, or may be used to support a tool T that is operated by a surgeon. The robot arm 40 may also be configured for collaborative/cooperative mode in which the operator may manipulate the robot arm 40. For example, the tooling end, also known as end effector, and/or tool T at the tooling end may be manipulated by the operator while supported by the robot arm 40;

The CAS controller 50 includes the processor(s) and appropriate hardware and software to run a computer-assisted surgery procedure in accordance with one or more workflows. The CAS controller 50 may include or operate the tracking module 60, the augmented reality module 70, and/or the robot driver 80 if present. Moreover, as described hereinafter, the CAS controller 50 may also drive the robot arm 40 through a planned surgical procedure, if the robot arm 40 is present;

The tracking module 60 is tasked with determining the position and/or orientation of the various relevant objects during the surgery procedure, such as the bone(s) and tool(s), using data acquired by the head-mounted tracking device 20 (e.g., video feed), the inertial sensor unit(s) 30 (e.g., angular rates of change) if present, and optionally the tracking device 90 if present. The position and/or orientation may be used by the CAS controller 50 to control the robot arm 40;

The augmented reality module 70 (a.k.a., mixed reality module) is provided to produce an augmented reality output to the operator, for instance for display in the head-mounted tracking device 20. The augmented reality module 70 may also produce other types of outputs, including a virtual reality output. The augmented reality module 70 may provide its output to displays other than head-mounted or wearable displays. For example, the augmented reality module 70 may produce an output for display on monitors of the CAS system 10, shown in FIG. 1 as interface I/F;

The robot driver 80 is tasked with powering or controlling the various joints of the robot arm 40, if present, based on operator demands or on surgery planning.

Other components, devices, systems, may be present, such as surgical instruments and tools T, the interfaces I/F such as displays, screens, computer station, servers, and the like etc.

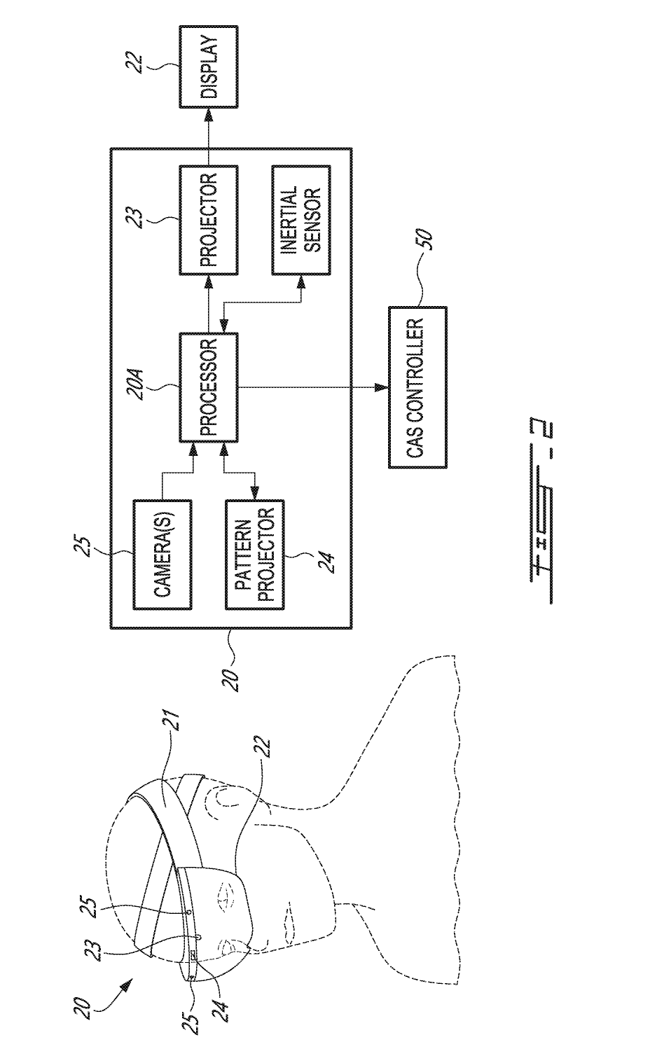

Referring to FIG. 2, a schematic example of the head-mounted tracking device 20 is provided. The head-mounted tracking device 20 may be as described in U.S. Patent No. 10,687,568, the contents of which are incorporated herein by reference, or may have other configurations. The head-mounted tracking device 20 described as a surgical helmet assembly in U.S. Patent No. 10,687,568 is well suited to be used in an augmented reality setting by its configuration. The head-mounted tracking device 20 may have a head enclosure 21 shaped to encircle a head of an operator. The head enclosure 21 may be straps, a rim, a helmet, etc. A face shield 22 may be mounted to a forehead or brow region of the head enclosure 21. The face shield 22 may be transparent to allow see-through vision by a user, but with the option of serving as a screen for augmented reality. Other components of the head-mounted tracking device 20 may include stabilizers, head band, a ventilation system with fan and vents, a light source, a rechargeable power source (e.g., a battery) etc.

The head-mounted tracking device 20 may consequently include a processor 20A and components to produce a mixed reality session. For instance, the head-mounted tracking device 20 may have an integrated projector 23 that may project data on the face shield 22, in a manner described below. Alternatively, the face shield 22 may be a screen having the ability to display images. As an example, the head-mounted tracking device 20 may be a HoloLens®. In an embodiment, the face shield 22 is a display-like unit of the type that may be used in virtual reality, with camera(s) therein to create a mixed reality output using camera footage, such as an Oculus Rift®, smartphone with head support, etc. The head-mounted tracking device 20 may include one or more orientation sensors, such as inertial sensor unit(s) (e.g., shown as 30), for an orientation of the head-mounted tracking device 20 to be known and tracked.

According to an embodiment, the head-mounted tracking device 20 is equipped to perform optical tracking of the patient tissue B, instruments T and/or robot arm 40, from a point of view (POV) of the operator. The head-mounted tracking device 20 may therefore have one or more imaging devices or apparatuses, to capture video images of a scene, i.e., moving visual images, a sequence of images over time. In a variant, the video images are light backscatter (a.k.a. backscattered radiation) used to track objects. In the present disclosure, the head-mounted tracking device 20 may be used to track tools and bones so as to provide navigation data in mixed reality to guide an operator based on surgery planning. Backscattered radiation can also be used for acquisition of 3D surface geometries of bones and tools.

The head-mounted tracking device 20 may produce structured light illumination for tracking objects with structured light 3D imaging. In structured light illumination, a portion of the objects is illuminated with one or multiple patterns from a pattern projector 24 or like light source. Structured light 3D imaging is based on the fact that a projection of a line of light from the pattern projector 24 onto a 3D shaped surface produces a line of illumination that appears distorted as viewed from perspectives other than that of the pattern projector 24. Accordingly, imaging such a distorted line of illumination allows a geometric reconstruction of the 3D shaped surface. Imaging of the distorted line of illumination is generally performed using one or more cameras 25 (including appropriate components such as e.g., lens(es), aperture, image sensor such as CCD, image processor) which are spaced apart from the pattern projector 24 so as to provide such different perspectives, e.g., triangulation perspective. In some embodiments, the pattern projector 24 is configured to project a structured light grid pattern including many lines at once as this allows the simultaneous acquisition of a multitude of samples on an increased area. In these embodiments, it may be convenient to use a pattern of parallel lines. However, other variants of structured light projection can be used in some other embodiments.

The structured light grid pattern can be projected onto the surface(s) to track using the pattern projector 24. In some embodiments, the structured light grid pattern can be produced by incoherent light projection, e.g., using a digital video projector, wherein the patterns are typically generated by propagating light through a digital light modulator. Examples of digital light projection technologies include transmissive liquid crystal, reflective liquid crystal on silicon (LCOS) and digital light processing (DLP) modulators. In these embodiments, the resolution of the structured light grid pattern can be limited by the size of the emitting pixels of the digital projector. Moreover, patterns generated by such digital display projectors may have small discontinuities due to the pixel boundaries in the projector. However, these discontinuities are generally sufficiently small that they are insignificant in the presence of a slight defocus. In some other embodiments, the structured light grid pattern can be produced by laser interference. For instance, in such embodiments, two or more laser beams can be interfered with one another to produce the structured light grid pattern wherein different pattern sizes can be obtained by changing the relative angle between the laser beams.

The pattern projector 24 may emit light that is inside or outside the visible region of the electromagnetic spectrum. For instance, in some embodiments, the emitted light can be in the ultraviolet region and/or the infrared region of the electromagnetic spectrum such as to be imperceptible to the eyes of the medical personnel. In these embodiments, however, the medical personnel may be required to wear protective glasses to protect their eyes from such invisible radiations, and the face shield 22 may have protective capacity as well. As alternatives to structured light, the head-mounted tracking device 20 may also operate with laser rangefinder technology or triangulation, as a few examples among others.

The head-mounted tracking device 20 may consequently include the cameras 25 to acquire backscatter images of the illuminated portion of objects. Hence, the cameras 25 capture the pattern projected onto the portions of the object. The cameras 25 are adapted to detect radiations in a region of the electromagnetic spectrum that corresponds to that of the patterns generated by the light projector 24. As described hereinafter, the known light pattern characteristics and known orientation of the pattern projector 24 relative to the cameras 25, are used by the tracking module 60 to generate a 3D geometry of the illuminated portions, using the backscatter images captured by the camera(s) 25. Although a single camera spaced form the pattern projector 24 can be used, using more than one camera 25 may increase the field of view and increase surface coverage, or precision via triangulation. The head-mounted tracking device 20 is shown as having a pair of cameras 25 is used.

The head-mounted tracking device 20 may also have one or more filters integrated into either or both of the cameras 25 to filter out predetermined regions or spectral bands of the electromagnetic spectrum. The filter can be removably or fixedly mounted in front of any given camera 25. For example, the filter can be slidably movable into and out of the optical path of the cameras 25, manually or in an automated fashion. In some other embodiments, multiple filters may be periodically positioned in front of a given camera in order to acquire spectrally resolved images with different spectral ranges at different moments in time, thereby providing time dependent spectral multiplexing. Such an embodiment may be achieved, for example, by positioning the multiple filters in a filter wheel that is controllably rotated to bring each filter in the filter wheel into the optical path of the given one of the camera 25 in a sequential manner.

In some embodiments, the filter can allow transmittance of only some predetermined spectral features of objects within the field of view, captured either simultaneously by the head-mounted tracking device 20 or separately by the secondary tracking device 90, so as to serve as additional features that can be extracted to improve accuracy and speed of registration.

More specifically, the filter can be used to provide a maximum contrast between different materials which can improve the imaging process and more specifically the soft tissue identification process. For example, in some embodiments, the filter can be used to filter out bands that are common to backscattered radiation from typical soft tissue items, the surgical structure of interest, and the surgical tool(s) such that backscattered radiation of high contrast between soft tissue items, surgical structure and surgical tools can be acquired. Additionally, or alternatively, where white light illumination is used, the filter can includes band pass filters configured to let pass only some spectral bands of interest. For instance, the filter can be configured to let pass spectral bands associated with backscattering or reflection caused by the bones, the soft tissue while filtering out spectral bands associated with specifically colored items such as tools, gloves and the like within the surgical field of view. Other methods for achieving spectrally selective detection, including employing spectrally narrow emitters, spectrally filtering a broadband emitter, and/or spectrally filtering a broadband imaging detector (e.g., the camera 25), can also be used. Another light source may also be provided on the head-mounted tracking device 20, for a secondary tracking option, as detailed below. It is considered to apply distinctive coatings on the parts to be tracked, such as the bone and the tool, to increase their contrast relative to the surrounding soft tissue.

In accordance with another embodiment, the head-mounted tracking device 20 may include a 3D camera(s), also shown as 25, to perform range imaging, and hence determine position data from the captured images during tracking – FIG. 2 showing two of such cameras 25 to enhance a depth perception. The expression 3D camera is used to describe the camera’s capability of providing range data for the objects in the image or like footage it captures, but the 3D camera may or may not produce 3D renderings of the objects it captures. In contrast to structured light 3D imaging, range tracking does not seek specific illumination patterns in distance calculations, but relies instead on the images themselves and the 3D camera’s capacity to determine the distance of points of objects in the images. Stated differently, the 3D camera for ranging performs non-structured light ranging, and the expression “ranging” is used herein to designate such non-structured light ranging. Such range tracking requires that the 3D camera be calibrated to achieve suitable precision and accuracy of tracking. In order to be calibrated, the head-mounted tracking device 20 may use a known visual pattern in a calibration performed in situ, at the start of the tracking, and optionally updated punctually or continuously throughout the tracking. The calibration is necessary to update the camera acquisition parameters due to possible lens distortion (e.g., radial, rotational distortion), and hence to rectify image distortion to ensure the range accuracy. Moreover, as described herein, tracking tokens with recognizable patterns (e.g., QR codes) may be used, with the patterns being used to determine a point of view (POV) of the cameras 25 of the head-mounted tracking device 20, via perspective deformation.

In a variant, the head-mounted tracking device 20 only has imaging capacity, for instance through cameras 25 (of any type described above), optionally pattern projector 24, without other components, such as face shield 22, etc.

Still referring to FIG. 1, the tracker device 30 provided in order to visually track the trackers A if present. The tracker device 30 may be embodied by an image capture device, capable of illuminating its environment, for compatibility with the trackers A. In a variant, the tracker device 30 may have two (or more) points of view, such that triangulation can be used to determine the position of the trackers A in space, i.e., the coordinate system of the surgical procedure. The tracker device 30 may emit light, or use ambient light, to observe the trackers A from its points of view, so as to determine a position of the trackers A relative to itself. By knowing the geometry of the arrangements of trackers A, the tracker device 30 can produce navigation data enabling the locating of objects within the coordinate system of the surgical procedure. In an embodiment, the tracker device 30 is of the type known as the Polaris products by Northern Digital Inc., known as NavitrackERs®.

As per another embodiment, as discussed above in the embodiments without the trackers A, the tracker device 30 may be a stereoscopic camera, a 3D camera, a motion detection camera, etc. Thus, without trackers A, the tracker device 30 may have the capacity to perform range imaging, and hence determine position data from the captured images during tracking. The expression 3D camera is used to describe the camera’s capability of providing range data for the objects in the image or like footage it captures, but the 3D camera may or may not produce 3D renderings of the objects it captures. In contrast to structured light 3D imaging, range tracking does not seek specific illumination patterns in distance calculations, but relies instead on the images themselves and the 3D camera’s capacity to determine the distance of points of objects in the images. Stated differently, the 3D camera for ranging performs non-structured light ranging, and the expression “ranging” is used herein to designate such non-structured light ranging. Such range tracking requires that the 3D camera be calibrated to achieve suitable precision and accuracy of tracking. In order to be calibrated, the tracking device 40 may use a known visual pattern in a calibration performed in situ, at the start of the tracking, and optionally updated punctually or continuously throughout the tracking. The calibration is necessary to update the camera acquisition parameters due to possible lens distortion (e.g., radial, rotational distortion), and hence to rectify image distortion to ensure the range accuracy. Moreover, as described herein, tracking tokens with recognizable patterns may be used, with the patterns being used to determine a point of view (POV) of the tracking device 30, via perspective deformation.

In FIG. 5, the tracker A is shown as having a support having trackable elements. In a variant, the exemplary tracker A is of the type arecognizable through detection by the tracker device 30 (FIG. 1). The tracker A may thus be known as trackable elements, markers, navigation markers, active sensors (e.g., wired or wireless) that may for example include infrared emitters. In a variant, the tracker A includes a support upon which are mounted passive retro-reflective elements, that reflect light. The elements are arranged in a known geometry, which geometry must differ from the geometry of the tracker 30 that is on the reaming device 15. Again, the elements may be retro-reflective tokens, patches, spheres, active elements such as LEDs, etc. In an example, the tracker A may be as described in U.S. Patent No. 8,386,022 and may thus be known as a multifaceted tracker.

Again, while the tracker A is shown as being of optical nature, it is considered to use different tracking modalities as an alternative to the trackers A. For example, it is contemplated to place a recognizable token, e.g., QR code, token, pattern or the like, on an object to be tracked. The tracking may then be performed by a camera unit, such as a 3D camera, that has suitable resolution to track the object within the precision requirements of computer-assisted surgery. The 3D camera may also perform the tracking without such QR code or equivalent. The 3D camera may be used to generate a model of the reaming device 15, i.e., a virtual 3D model, which model is then tracked in position and orientation. As an alternative to a full 3D model, the 3D model may use prominent visual features of the registering device 20 to track it.

Referring to FIGS. 1 and 5, the robot arm 40 may stand from a base, for instance in a fixed relation relative to the operating-room (OR) table supporting the patient, whether it is attached or detached from the table. The robot arm 40 has a plurality of joints and links, of any appropriate form, to support a tool T that interfaces with the patient. The end effector or tool head may indeed optionally incorporate a force/torque sensor for collaborative/cooperative control mode, in which an operator manipulates the tool T at the end of the robot arm 40. The robot arm 40 is shown being a serial mechanism, arranged for the tool head to be displaceable in a desired number of degrees of freedom (DOF). For example, the robot arm 40 controls 6-DOF movements of the tool head, i.e., X, Y, Z in the coordinate system, and pitch, roll and yaw. Fewer or additional DOFs may be present. For simplicity, only a generic illustration of the joints and links is provided, but more joints of different types may be present to move the tool head in the manner described above. The joints are powered for the robot arm 40 to move as controlled by the CAS controller 50 in the six DOFs, and in such a way that the position and orientation of the tool head in the coordinate system may be known, for instance by readings from encoders on the various joints. Therefore, the powering of the joints is such that the tool head of the robot arm 40 may execute precise movements, such as moving along a single direction in one translation DOF, or being restricted to moving along a plane, among possibilities. Such robot arms 40 are known, for instance as described in United States Patent Application Serial no. 11/610,728, and incorporated herein by reference. The head-mounted tracking device 20 and/or tracking device 30 may be used for the tracking of the end effector of the robot arm 10, or other systems such as inertial sensor systems.

Still referring to FIG. 1, the CAS controller 50 is shown in greater detail relative to the other components of the robotized CAS system 10. The CAS controller 50 has a processor unit 51 (one or more processors) and a non-transitory computer-readable memory 52 communicatively coupled to the processing unit 51 and configured for executing computer-readable program instructions executable by the processing unit 51 to perform some functions, such as tracking the patient tissue and tools, using the camera feed from the head-mounted tracking device 20 and/or from the tracking device 30. The CAS controller 50 may also control the movement of the robot arm 40. The CAS system 10 may comprise various types of interfaces I/F, for the information to be provided to the operator. In addition to the head-mounted tracking device 20, the interfaces I/F may include a monitor and/or screens including wireless portable devices (e.g., phones, tablets), audio guidance, LED displays, among many other possibilities. For example, the interface D includes a graphic-user interface (GUI) operated by the system 10. The CAS controller 50 may also display images captured by the cameras 25 of the head-mounted tracking device 20 and/or tracking device 30, for instance to be used in the collaborative/cooperative control mode of the system 10, or for visual supervision by the operator of the system 10, with augmented reality for example. The CAS controller 50 may drive the robot arm 40, if present, in performing the surgical procedure based on the surgery planning achieved pre-operatively. The CAS controller 50 may run various modules, in the form of algorithms, code, non-transient executable instructions, etc, in order to operate the CAS system 10 in the manner described herein. The CAS controller 50 may be part of any suitable processor unit(s), such as a personal computer or computers including laptops and desktops, tablets, server, cloud, etc.

The tracking module 60 may be a subpart of the CAS controller 50, or an independent module or system. The tracking module 60 receives from the head-mounted tracking device 20 and the tracking device 30 (if present) the video feed of the surgical scene, e.g., as backscatter images of the objects. The tracking module 60 may also concurrently receive tracking data (e.g., orientation data) from the robot arm 40. In an embodiment, as the system 10 performs real-time tracking, the video images and the orientation data are synchronized, as they are obtained and processed simultaneously. Other processing may be performed to ensure that the video footage and the orientation data are synchronized.

The tracking module 60 processes the video images to track one or more objects, such as a bone, an instrument, etc. The tracking module 60 may determine the relative position of the objects, and segment the objects within the video images. In a variant, the tracking module 60 may process the video images to track a given portion of an object, that may be referred to as a landmark. The landmark may be different parts of the objects, objects on the objects, tracking tokens with recognizable patterns, etc.

The tracking module 60 may also be provided with models of the objects to be tracked. For example, the tracking module 60 may track bones and tools, and hence uses virtual bone models and tool models. The bone models may be acquired from pre-operative imaging (e.g., MRI, CT-scans), for example in 3D or in multiple 2D views, including with 2D X-ray to 3D bone model technologies. The virtual bone models may also include some image processing done preoperatively, for example to remove soft tissue or refine the surfaces that will be exposed and tracked. The virtual bone models may be of greater resolution at the parts of the bone that will be tracked during surgery, such as the knee articulation in knee surgery. The bone models may also carry additional orientation data, such as various axes (e.g., longitudinal axis, mechanical axis, etc). The bone models may therefore be patient specific. It is also considered to obtain bone models from a bone model library, with the data obtained from the video images used to match a generated 3D surface of the bone with a bone from the bone atlas. The virtual tool models may be provided by the tool manufacturer, or may also be generated in any appropriate way so as to be a virtual 3D representation of the tool(s). In a variant, the bone models do not include a full bone, but may only include a bone surface of a portion of a bone, such as for example the portion of the bone that is being resected. Such partial bone model, referred to herein as bone model, 3D bone model, virtual bone model, may include additional data, such as one or more axes. For example, in the case of a femur in knee surgery, the bone model may include a distal femur portion only. The bone model may also include a mechanical axis of the femur, that may be acquired pre-operatively via imaging, or intraoperatively through a calibration procedure (e.g., tracked movements of the femur relative to the pelvis). For a complete model, in a variant in which the model of the bone is partial, a bone atlas may be used to find an equivalent bone model, i.e., with corresponding features, which equivalent bone model may be merged with the partial bone model of the patient. All of the above variants of the bone model apply to any other bone described herein.

In a variant, the tracking module 60 may generate 3D models using the video images. For example, if the tracking module 60 can have video images of a tool, from 360 degrees, it may generate a 3D model that can be used for subsequent tracking. This intraoperative model may or may not be matched with pre-existing or pre-operative model of the tool.

Additional data may also be available, such as tool orientation (e.g., axis data and geometry). By having access to bone and tool models, the tracking module 60 may recognize an object in the image processing and/or may obtain additional information, such as the axes related to bones or tools. The image processing by the tracking module 60 may be assisted by the presence of the models, as the tracking module 60 may match objects from the video images with the virtual models.

In a variant, the objects used as landmarks are parts of the bone and of the tool that are visible from the head-mounted tracking device 20. Stated differently, as the operator has a direct and proximal view of the surgical site, e.g., the bone being resected and the tool performing the resection, the footage from the POV of the head-mounted tracking device 20 is used by the tracking module 60 to navigate the tool T relative to the bone B. Despite the variation in POV of the camera(s) 25, the tracking module 60 uses the known dimensions of a landmark to track the objects in a referential system. The body of the tools T may for example be used as a basis for the tracking, as explained above, by way of the model of the body of the tool T.

Optionally, it is considered to provide specific detectable landmarks on the tool(s) or bones to ensure the detectable landmarks will be properly imaged and detected by the tracking module 60. Indeed, in some instances when the view of some of the objects is limited, trackers A may used. As another example, tokens with given patterns, such as QR-code like labels, may be provided on the objects, such as on the bones or on the tools. In an embodiment, the tokens have an image that may be preprogrammed, or whose dimensions, are known or accessible by the tracking module 60. Accordingly, by seeing such tokens in the video images, the tracking module 60 may locate the objects in a spatial coordinate system. Again, the POV for the video images may move, with the objects serving as a referential for the tracking. The referential system may be that of the robot arm 20 (if present), with the camera tracking providing positional information for the referential system.

In matching the recognizable pattern and/or the 3D geometry to the bone models and tool models with the video images, the tracking module 60 may reduce its computation using different strategies. The bone model(s) B may have higher resolution for the parts of the bone that will be altered during surgery. The remainder of the bone may be limited to information on landmarks, such as axis orientation, center of rotation, midpoints, etc. A similar approach may be taken for the tool models C, with the focus and higher detail resolution being on parts of the tools that come into contact with the bone.

Moreover, considering that the camera(s) 25 may interrupt its line of sight with the object, the video feed from the tracker device 30 may complement that from the camera(s) 25 and/or supersede the video feed from the camera(s) 25. In another embodiment, the tracker device 30 is the primary tracking camera using any of the technologies described above for the head-mounted tracking device 20. Therefore, the tracking module 60 continuously updates the position and/or orientation of the patient bones and tools in the coordinate system using the video feed from the camera(s) 25 and/or tracking device 30 to track objects in position and orientation.

In an embodiment with structured light projection, the tracking module 60 receives the backscatter images from the camera(s) 25 or from the tracking device 30, as a result of the structured light projection from the projector 24. In another embodiment, the tracking module 60 receives the video images from the camera 25 in a depth camera configuration, and may ensure take steps to calibrate the camera(s) 25 or tracking device 90 for ranging to be done from the acquired images. An initial calibration may be done using a calibration pattern, such as that in tokens. The calibration pattern is placed in the light of sight of the camera(s) 25 or tracking device 90 such that it is imaged. The calibration pattern is any appropriate shape and configuration, but may be a planar recognizable pattern with high contrast, or given landmarks of a bone, or geometry of tool. Other items can be used for the calibration, including the body of a tool T, whose geometry may be programmed into or may be accessed by the tracking module 60. This includes a preoperative model of the tool(s) that may be merged with live tracking data for the tool. The tracking module 60 stores a virtual version of the calibration pattern, including precise geometrical data of the calibration pattern. The tracking module 60 therefore performs a correspondence between imaged and virtual calibration patterns. The correspondence may entail calculating the mapping function between landmarks on the planar imaged calibration pattern and the virtual calibration pattern. This may include a projection of the calibration patterns on one another to determine the distortion characteristics of the images of the camera(s) 25 or tracking device 30, until the rectification values are determined by the tracking module 60 to correct the images of camera. This calibration may be repeated punctually through the procedure, for instance based on the camera updating requirements. It may require that the camera be used in conjunction with a calibration reflective surface whose position and orientation relative to the camera is known. The calibration may be automatically performed by the CAS system 10.

The tracking module 60 may therefore perform a 3D geometry image processing, using the known patterns of structured light, or calibrated camera images, video feed, etc, along with the known shape of the virtual bone model(s) and/or tool model(s), optionally with QR tokens or trackers A, and generate 3D images from the tracking, using for examples the pre-operative models. Moreover, a generated 3D geometry may be located in the X, Y, Z, coordinate system using the tracking of landmarks on the bones or tools to set the coordinate system on the bones. Therefore, the tracking module 60 may generates an image or 3D geometry of the landmarks on the object(s) being illuminated. Then, using the virtual models and/or of the bone(s) and tool(s), respectively, the tracking module 60 can match the image or 3D geometry with the virtual models of the landmarks. Consequently, the tracking module 60 determines a spatial relationship between the landmarks being imaged and the preoperative 3D models, to provide a dynamic (e.g. real time or quasi real time) intraoperative tracking of the bones relative to the tools, in spite of tool portions and bone surfaces not being visible from the POV of the operator.

In an embodiment, the position and orientation of the surgical tool calculated by the tracking module 60 may be redundant over the tracking data provided by the robot driver 80 and robot arm sensors, if the robot arm 40 is used. However, the redundancy may assist in ensuring the accuracy of the tracking of the surgical tool. For example, the redundancy is used as a safeguard against disruption of the line of sight between the head-mounted tracking device 20 and the surgical site, for instance if the operator looks away. The redundancy may also allow the reduction of frequency of image processing for the surgical tool. Also, the tracking of the tool using the tracking module 60 may be used to detect any discrepancy between a calculated position and orientation of the surgical tool T through the sensors on the robot arm 40, and the actual position and orientation of the surgical tool. For example, an improper mount of the tool T into the chuck of the robot arm 40 could be detected from the output of the tracking module 60, when verified with the position and orientation from the robot driver 80 (e.g., obtained from the encoders on the robot arm 40). The operator may be prompted to verify the mount, via the interface I/F or head-mounted tracking device 20.

The camera 25 and/or tracking device 30 may continuously capture images, for the tracking module 60 to perform a continuous tracking of the objects. The terms video feed, image feed, video, may be used to describe the capture of images by the head-mounted tracking device 20, and optionally by the tracking device 30, and entails a capture of frames over a time period, in contrast to a single image capture. The frequency of capture may vary according to different factors. For example, there may be different phases during the surgical workflow, some in which the tracking requires a more dynamic update (i.e., higher frequency), and some in which tracking updates are less important. Another factor that may affect the image capture frequency is the fixed relation of the objects. For example, once the tracking module 60 identifies a landmark and tracks a bone from the images, the frequency capture by the camera 25 and/or tracking device 30 may be reduced if the bone is fixed or if no maneuvers are performed, if the bone alterations have not yet begun. Also, when both a tool and a bone are tracked, the frequency capture may be reduced when the tool and the bone are spaced from one another by a given distance, and increased as the proximity between the tool and the bone is increased. The tracking module 60 may drive the camera 25 and/or tracking device 30 in order to control the frequency. For example, the tracking module 60 may adapt the frequency using the surgical planning, e.g., anticipating upcoming steps in the workflow, etc. The tracking module 60 may consequently toggle between a lower frequency capture mode and a higher frequency capture mode, for example. The lower frequency capture mode may be in instances in which the tool is at a given distance from the bone, and is not driven to alter the bone. The lower frequency capture mode may also be operated when the objects are in a fixed relation relative to one another. Other modes are contemplated. The tracking module 60 may output the data directly on the interfaces I/F.

The augmented reality module 70 may be present in the CAS controller 50 and may produce an augmented reality (AR) output to the operator, for instance for display in the head-mounted tracking device 20. The augmented reality module 70 may also produce other types of outputs, including a virtual reality output. The augmented reality module 70 may provide its output to displays other than head-mounted tracking device 20. For example, the augmented reality module 70 may produce an output for display on monitors of the CAS system 10.

Still referring to FIG. 1, the CAS controller 50 may have the robot driver module 80, if a robot arm 40 is present in the CAS system 10. The robot driver module 80 is tasked with powering or controlling the various joints of the robot arm 40. There may be some force feedback provided by the robot arm 40 to avoid damaging the bones. The robot driver module 80 may perform actions based on a surgery planning. The surgery planning may be a module programmed specifically for any given patient, according to the parameters of surgery desired by an operator such as an engineer and/or surgeon. The parameters may include geometry of selected, planned bone cuts, planned cut depths, sequence or workflow of alterations with a sequence of surgical steps and tools, tools used, etc.

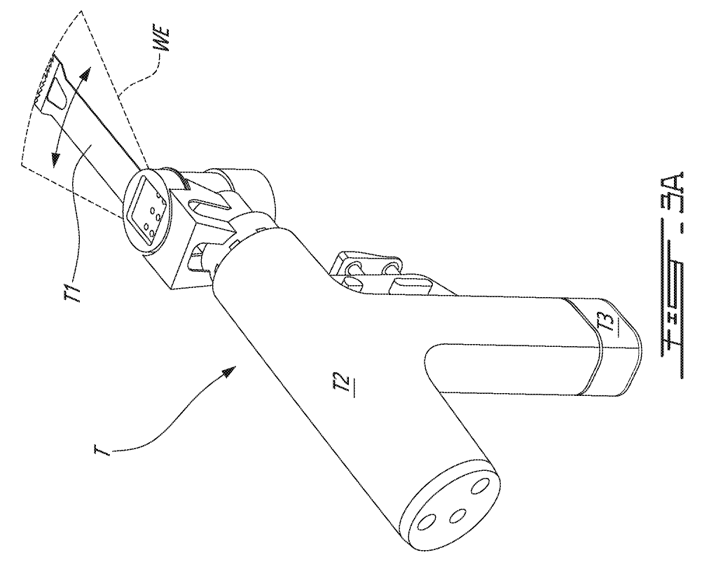

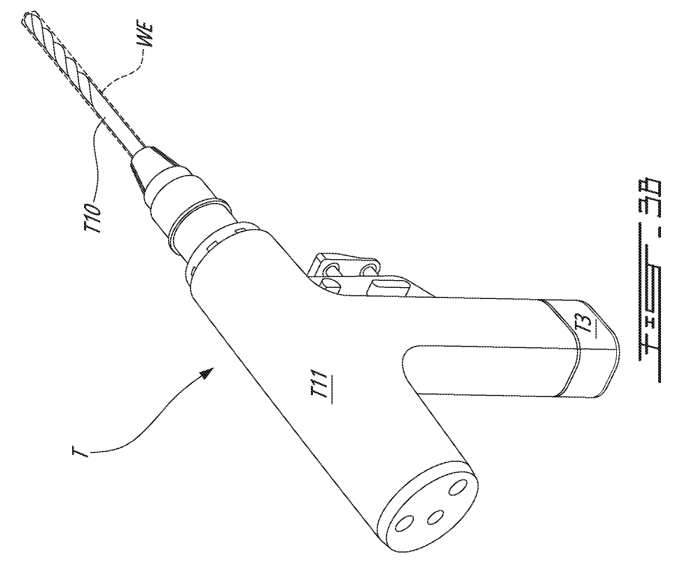

Two distinct tools T are shown in FIGS. 3A and 3B. In FIG. 3A, the tool T is an oscillating saw having a blade T1 at a working end of the tool body T2. The tool body T2 is of the type that has a handgun shape, with actuating triggers. For example, the oscillating saw may be part of the X Series Power System^{TM} ensemble, by Zimmer Biomet. The blade T1 has its cutting edge at its free end, and performs a back and forth reciprocating motion, such that it covers a working envelope. The arcuate path of the working envelope WE is that along which the blade T1 moves to resect the bone. In FIG. 3B, the tool T is a drill having a drill bit T10 at a working end of the tool body T11. The drill bit T10 has a working envelope WE. The tool body T11 is also of the type that has a handgun shape, with actuating triggers. FIGS. 3A and 3B are representative of examples of tools that are used to perform alterations on bones, and multiple other tools could also be used and tracked with the CAS system 10. The working envelope WE of the tools T is relevant information, in that it is representative of the alterations on a bone. Accordingly, it may be tracked by the tracking module 60. In a variant, a calibration may be performed so as to model the working envelope WE. For example, the oscillating saw T of FIG. 3A may be actuated while being imaged, for the image processing to generate the working envelope WE from the amplitude of movement of the blade T1. The working envelope WE could also be accessible as a virtual model, for instance from the manufacturer. In any case, the working envelope WE may be relative to the body T2 (same principle applying to the drill T of FIG. 3B), with the body T2 having a fixed geometry and being viewable from the POV of the operator wearing the head mounted tracking device 20 during surgery.

The tools T of FIGS. 3A and 3B may both have a power pack, shown as T3. The power pack T3 may include a battery for wireless operation of the tools T. Alternatively, the power pack T3 could be connected to an electric power source via a wire, or may be connected by tubing or piping to another motive source, such as hydraulic or pneumatic pressure network. The power pack T3 may include a controller unit, by which the tools T may be shut off, with the controller unit being communicatively coupled to the CAS controller 50 of the CAS system 10 (or to the processor 20A of the head-mounted tracking device 20), to receive commands therefrom, optionally. Thus, the power pack T3 (or any other part of the tool T) may have wireless communication capacity. Therefore, if one of the modules of the CAS system 10, such as the tracking module 60, identifies a stop condition, the CAS controller 50 may stop the operation of the tool. The stop condition may include the proximity of the working end WE of the tool T with soft tissue, a threshold zone around soft tissue that must not be impinged by the working end WE, the hidden boundary of a surface of the bone.

Referring to FIG. 4A, a cutting block that may be used with the CAS system 10 is generally shown at 90. The cutting block 90 may be referred to as 4-in-1 femoral instrumentation, 4-in-1 cutting guide, 4-in-1 cutting block. The cutting block 90 has a block body 91 that has an abutment plane 91A configured to be abutted against a distal cut of a femur. The expression 4-in-1 refers to the fact that the cutting block 90 may be used to guide in the resection of four different cuts on the distal femur, i.e., an anterior cut, an anterior chamfer cut, a posterior chamfer cut, and a posterior cut. These cuts are typically made for an implant having a conforming geometry to be fitted in the distal femur once the cuts have been made. Accordingly, some slots are defined in the cutting block 90, and are jointly referred to as the slots 92. The slots 92 are configured to receive therein a flat saw blade such as that shown as T1 in FIG. 3A. The slots 92 may be said to correspond to a cutting plane of the cutting block 90. Tolerances are such that the use of the slots 92 with an appropriately sized saw blade results in a highly predictable location of the cut, based on a position of the cutting planes in the cutting block. The slots 92 are illustrated as slot 92A for the anterior cut, slot 92AC for the anterior chamfer cut, slot 92PC for the posterior chamfer cut, and slot 92P for the posterior cut. Some other cutting blocks 90 could also be used as well, for instance fewer slots, or like saw guides, such as a cutting block featuring only slots for the anterior cut and the posterior cut, i.e., two cutting planes. The cutting block 90 may also include various bores 93 to be secured onto the femur, again with its abutment plane 91A being against the distal cut plane of the femur.

Referring to FIG. 4B, parts of a virtual model of the cutting block 90 are shown. The virtual model may be a ROM file that includes geometrical data representative of the cutting block 90. For example, the virtual model of the cutting block 90 may include the location of the cutting planes associated with the slots 92, relative for example to a plane corresponding to the abutment plane 91A, the abutment plane 91A shown as abutted against the distal cut of the femur. In the embodiment of FIGS. 4 and 5, i.e., a 4-in-1 cutting block, the virtual model may include cutting plane 1 correspond to the anterior cut, cutting plane 2 correspond to the posterior cut, cutting 3 corresponding to the posterior chamfer cut, and cutting plane 4 corresponding to the anterior chamfer cut. In a variant, the cutting block 90, and the virtual model of the cutting block 90, correspond to a given implant, i.e., the cutting block 90 is used specifically for an implant of a given identity and size. The cutting block 90 is used to prepare the femur to receive the given implant, such that a well executed surgical procedure with the cutting block 90 may result in the precise and accurate fit of the implant on the resected bone. Often, the implant may be selected by an operator as a function of the patient needs, and the cutting block 90 may be rendered available as a function of the selection of the implant. Therefore, when the virtual model is used to navigate the positioning of the cutting block 90 on the bone, as described hereinafter, the virtual model is specific to a selection of implant.

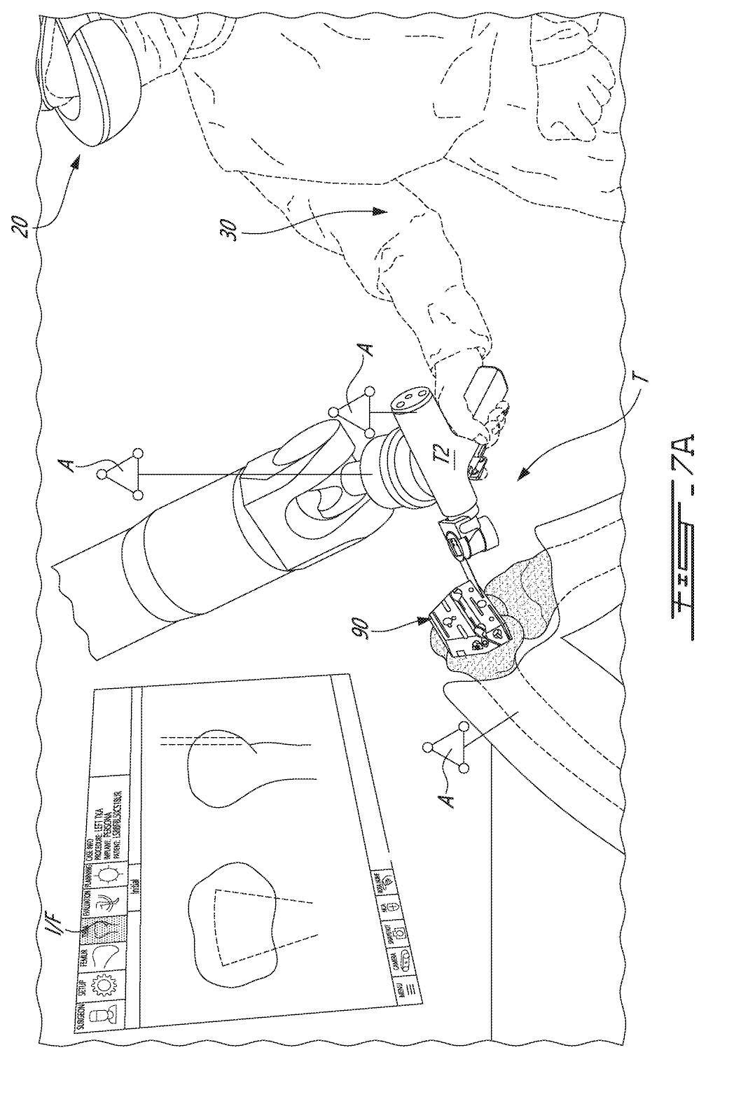

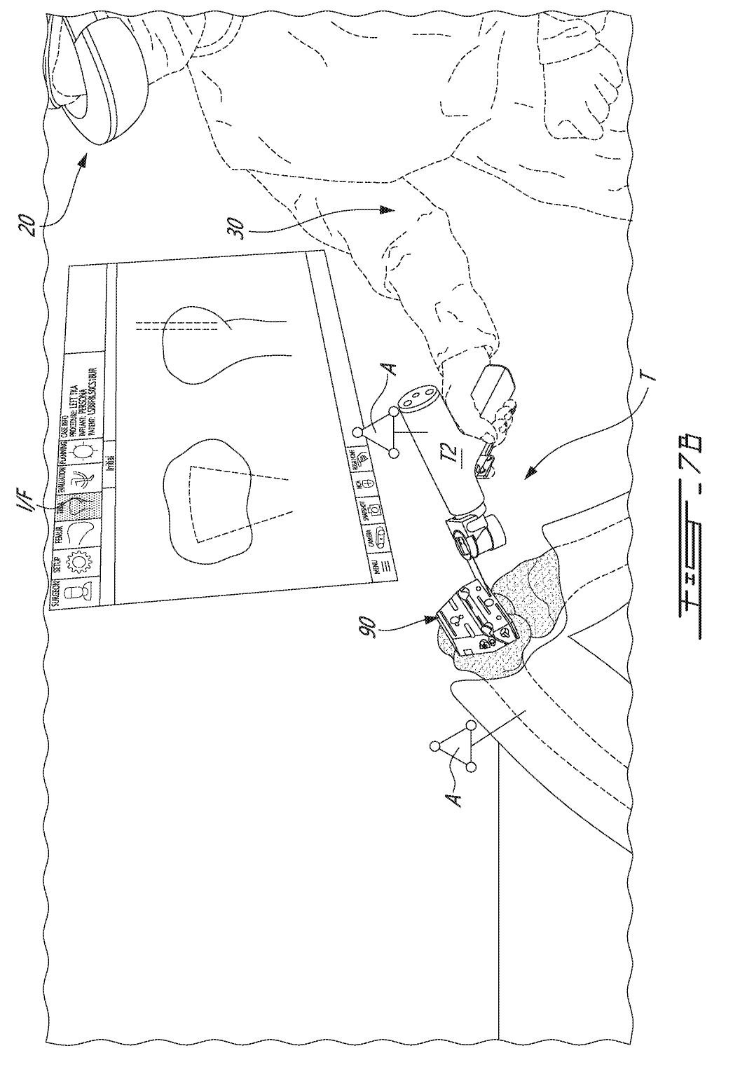

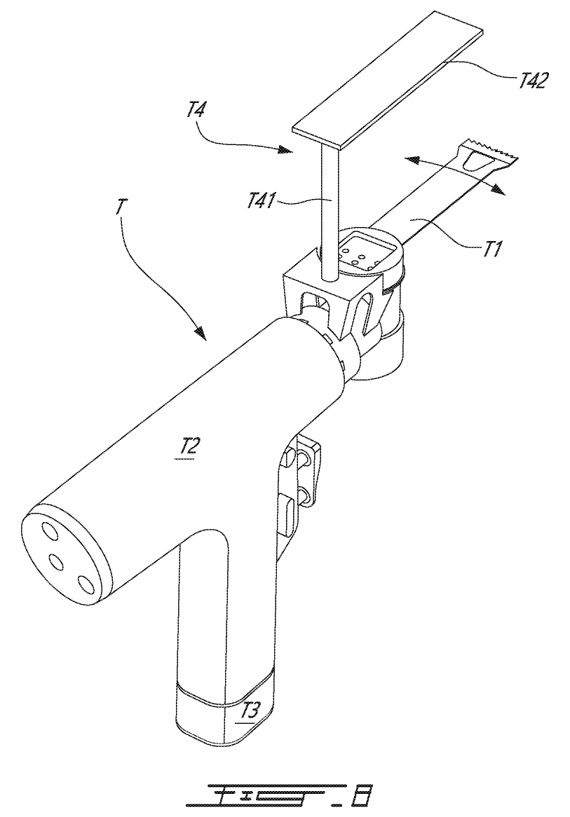

Referring now to FIG. 5, a contemplated use of the CAS system 10 is shown, as occurring during a method 100 for resecting a bone in robotized CAS, such as the resection of the distal femur to receive thereon a femoral component of a knee implant. Steps of the method 100 may be exemplified by FIGS. 6 and 7, in which a surgeon, healthcare professional or other operator has the head-mounted tracking device 20 with tracking technology as described above, with camera(s) 25 to perform point of view tracking. Optionally, though not shown, the tracking device 30 may be used as an alternative to the head-mounted tracking device 20, or in addition to it, with the trackers A as shown. The method 100 is described as having a series of steps. However, some of the steps may be sub-steps, some of the steps may be concurrent, some of the steps may be continuous while others may be discrete. Hence, the expression “step” should encompass such possibilities, based on the following description.

The robot arm 40 may be used in a collaborative mode, i.e., the operator manipulates the robot arm 40, with the robot arm 40 being actuated to assist the operator in the manipulations, to position the robot arm 40 as part of planning, etc. The robot arm 40 may also be used in an automated mode, in which movements of the robot arm 40 may be fully automated. As part of the modes, it may be the operator that actuates the tools T, such as a drill or a saw, while the robot arm 40 supports the tools T and holds their positions. In some instances, in spite of the stiffness and rigidity characteristics of the robot arm 40, side loads of power tools may cause some deflections in the robot arm 40, such as during bone alterations. The method 100 may be used to reduce any error caused by such deflections.

According to step 101 of FIG. 5, in order to navigate tools T, bones B and the robot arm 40, a referential system (a.k.a., frame of reference, coordinate system) is created. The referential system may be said to be common to the various objects used being navigated during the surgical procedure, i.e., it may be global to all of the tracked items, and its origin may be arbitrary (e.g., on the robot, on the bone, etc). In the example of the femur, at least the surface of the distal femur may be registered or digitized in the referential system. Additional information may be registered in the referential system, including bone axes (e.g., anatomical axis of the femur, mechanical axis of the femur), and similar data for other bones, including the tibia. Indeed, while the method 100 focuses on the distal femur in the present description, the method 100 may also be performed on the tibia, to resect the tibial plateau to add a tibial implant component. In a variant, a handshake approach may be taken, by recording points on the bone (i.e., femur) in the common referential system, using the robot arm 40. The robot arm 40 may for example have a registration pointer as end effector, and may be manipulated or controlled to touch various points on the surface of the bone to generate a model thereof. In a variant, pre-operative imaging of the bone may have been done for 3D model(s) of the bone to be available. The recordation of points may allow the CAS system 10 to merge the 3D bone model to the recorded points. Other variants include the generation in situ of the bone model, using appropriate imaging cameras, such as depth cameras. An in situ bone model may be merged with a cloud of points resulting from registration as described above. Depth cameras may be part of the head-mounted tracking device 20 or of the tracking device 30, or may be an additional device of the CAS system 10.

According to step 102 of FIG. 5, the robot arm 40 and the bone B are tracked in the common referential system. This may also be referred to as navigation of the robot arm 40 and of the bone B. Moreover, the tools T may be navigated. The tracking is performed by the tracking modality, e.g., the head-mounted tracking device 20, the tracking device 30, trackers A, and/or the position sensors in the robot arm 40, or others. The tracking provides position and orientation data for the objects in the common referential system. The tracking may be done continuously during the method 100, though this may include pauses, etc. However, it may be said that the tracking is continuous in that it is performed during manipulations of objects in the surgical field, in real-time and at a frequency allowing live tracking.

According to step 103 of FIG. 5, a model of an implant to be used for the patient is obtained. While this is shown as step 103, it may have been done before, including preoperatively. The model of the implant may include an identity thereof (e.g., identification number, size, geometrical data, virtual model, model type, etc). The model of the implant may have an impact on the resections made to the bone, and may have an impact on the selection of the various cutting blocks and other tools that will be used during surgery.

According to step 104 of FIG. 5, a virtual model of a distal cutting block to be used on the femur is obtained. The virtual model may include a cutting plane corresponding to a distal cut of the femur. The virtual model of the distal cutting block is dependent on the implant model, as the depth of the distal cut and/or location of other cuts may be as a function of the implant. In some instances, the step 104 is optional, as the robot arm 40 may be operated to perform the distal cut without the use of a distal cutting block, i.e., a cutting block used to perform the distal cut.

According to step 105 of FIG. 5, if a distal cutting block is used, the robot arm 40, having the distal cutting block supported at an end thereof, is controlled to place the distal cutting block in a desired location on the femur, such as on an anterior surface of the femur, using the model of the distal cutting block and the tracking of the robot arm 40 and of the bone B. The robot arm 40 may be operated to perform the distal cut using the distal cut guide, either automatically or by the actuation of the operator. The distal cut guide may be supported by the robot arm 40 during the resection, but it is also possible for the user to pin the distal cut guide to the bone, so as not to use the robot arm 40 during the resection of the distal cut. Alternatively, the robot arm 40 may be operated to perform the distal cut without the use of a distal cutting block, by the CAS system 10 controlling the robot arm 40. In both scenarios, the robot arm 40 may be controlled to drill a peg hole in the femur, such as at the entry point of the mechanical axis, if the implant is of the type that has a peg.

According to step 106 of FIG. 5, a virtual model of a cutting block to be used on the bone is obtained, the cutting block for example being that of FIG. 4A and the model having data similar to that of FIG. 4B. The virtual model of the cutting block may be obtained from a manufacturer file, etc, or may be generated in situ. The virtual model is for example a 3D model that includes slots corresponding to two or more cutting planes of the cutting block, relative to a base line that may for instance be the abutment plane 91A (FIG. 4A) of the cutting block 90, as the abutment plane 91A is laid onto the distal cut of the femur. The virtual model of the cutting block may include the anterior cut plane and the posterior cut plane, respectively shown as 1 and 2 in FIG. 4B. Additional planes of the virtual model of the cutting block may include the posterior chamfer cut plane 3 and the anterior chamfer cut plane 4 as shown in FIG. 4B.

According to step 107 of FIG. 5, the robot arm 40 is controlled, such as when it has the cutting block supported at an end thereof, to place the cutting block in a desired location on the bone, using the model of the cutting block and the tracking. The robot arm 40 is tracked such that the cutting block is positioned and oriented in a desired way, i.e., with the cutting planes precisely located where they should be, in the correct pose of the cutting block relative to the bone. The step 107 may include controlling the robot arm in the desired location against a distal cut of the femur, with proper position and orientation, again based on the cutting planes being precisely located, based on the tracking and on the model of the cutting block. This is shown in FIG. 6. Once the cutting block is appropriately located on the bone, as in FIG. 6, and as confirmed by the tracking and the virtual model, the cutting block 90 may be secured to the bone. One contemplated way to achieve this is by having the cutting block 90 pinned to the femur, using the tool set up of FIG. 3B. As illustrated in FIG. 4A, the cutting block 90 may have bores 93 configured to receive pins, screws, etc. As the robot arm 40 is active in maintaining the cutting block 90 in a desired location on the bone B, it may be the operator that performs the securing of the cutting block 90 on the femur F. Precautions must be taken to avoid any displacement caused by the position of pins, but the combination of the stiffness of the robot arm 40 with the friction of abutment between the cutting block 90 and the distal cut may prevent any such displacement. Moreover, the drilling operation may not be conducive to substantial side loads. Once the cutting block 90 is fixed to the bone, i.e., the femur in FIG. 6, the robot arm 40 may be separated from the cutting block 90 to perform other tasks.

According to step 108 of FIG. 5, the robot arm 40 may have a cutting tool supported at the end thereof, such as the tool of FIG. 3A with the blade T1. The robot arm 40 may position itself relative to the cutting block 90 for the blade T1 to be aligned with the slots and cutting planes. The robot arm 40 may be configured to actuate the tool for the blade T1 to resect the femur. In a variant, it is the operator that pulls the trigger to activate the tool, as in FIG. 7A. In either case, the fact that the cutting block 90 is anchored to the bone prevents any deflection of the robot arm 40 during the resection. As another possibility, as shown in FIG. 7B, the tool T and blade T1 may be operated in a freehand mode, i.e., with the tool T supported by the operator. In the freehand mode, the user relies on the cutting block 90 to be guided in resecting the bone based on the planning.

In one or more of the steps in which the bone is altered, and more particularly when the planes are cut using the distal cut guide and/or the cutting block 90, the CAS system 10 and method 100 may be operated to (1) validate that the cutting block is correctly posed onto the bone, and (2) protect soft tissue surrounding the bone. The CAS system 10 may calculate and continuously output a location of a working end WE of the surgical tool T2 relative to the bone, such as in a concealed condition of the working end WE of the surgical tool relative to the bone, as a bone altering operation occurs, such as plane resection, bone drilling, etc. This may occur for example in steps 104, 105 and/or 108. Therefore, the continuous tracking of the tool may be compared to the planned location of the planes that may used to place the cutting block on the bone. If a discrepancy is detected, the operator may be prompted to performed verification steps. As for the concealed condition of the working end WE, as shown in FIGS. 6, 7A and 7B, the CAS system 10 may therefore display the working end of the surgical tool relative to the bone, even though the working end is concealed or hidden, notably in the circumstances of a limited incision in minimally invasive surgery. This includes a display of the working envelope WE of the surgical tool, as shown in FIGS. 6, 7A and 7B. Therefore, the operator may have live virtual footage of the working end of the surgical tool relative to the bone penetrating the bone on an interface I/F. The CAS system 10 may hence detect stop conditions, to alarm the operator, and/or to stop the tool T2, for instance by indicating a proximity of the working end with a boundary of the bone. In parallel, the CAS system 10 may image or display the concealed working end of the surgical tool relative to the bone, such as on the head-mounted tracking device 20, e.g., in mixed reality on a face shield worn by the operator, or on other interfaces. The CAS system 10 may calibrate the tool(s) before the bone altering maneuvers, such as by obtaining video images of the surgical tool and its working end and processing same to size the model of the surgical tool. The tool T2 may be operated to image an amplitude of movement of the working end and size the amplitude of movement of the working end. In the variant of FIG. 7A, the robot arm 40 may be controlled as a function of a position and orientation of the tool and/or through the video images of a tool such as a cutting block 90 being processed. Thus, the CAS system 10 may continuously output the location of the robot arm 40 (or of a tool supported by the robot arm 40, such as the illustrated cutting block 90). Accordingly, tracking guidance may be output for the cut to be made, including an image of the concealed working end of the tool relative to the periphery of the bone. Tracking guidance may be to the robot arm 40 or to the surgeon or staff member. The CAS controller 50 may be provided with the geometry of the various tools used (and cutting guide 90 and blade T1 combination as in FIG. 7B), and the tool may be tracked relative to the referential system using the head-mounted device 20 and imaging capacity thereof, and/or tracker device 30. With the image processing capacity of the CAS system 10, the tracking module 60 may precisely calculate the spatial positioning of the concealed working end of the tool T relative to hidden surfaces of the bone, and provide a virtual live representation thereof, and this may include using the model of the cutting guide 90 to determine the specific boundary of the cutting plane relative to a model of the bone. The tracking module 60 may indeed combine the cutting plane(s) of the cutting guide 90 with the model of the bone to define a cutting envelope in which the cutting tool may be operated to alter the bone, and outside of which the cutting block should not be operated. The tracking module 60 may thus be configured to detect stop conditions, such as the proximity of soft tissue that could be damaged by the working end WE. The tracking module 60 may send alarm signals to the operator, image the proximity on the GUIs or like interfaces I/F, and shut off the tools T, to prevent substantial soft tissue damage. The tracking module 60 may also operate a deceleration profile for the working end WE to decelerate its movements, within a threshold zone in proximity to the contour of the bone, while navigating the cutting block 90.

Some or all of the steps described above may be repeated for a similar procedure to be performed on the tibia. This may occurred before the resection of the femur, or after. Moreover, the method 100 is not limited to knee surgery.

In the variant of the tool of FIG. 3A, tool T with saw blade T1 may be equipped with a patient-specific cut guide T4, also known as PSI cut guide, in that it has contour matching surface(s) to abut a bone in a predetermined way. The cut guide T4 is provided to assist in locating the cut plane for the surgeon or other operator manipulating the tool T, and thus to ensure that the tool T follows a desired trajectory, such as along a plane for the blade T1. The PSI cut guide T4 (or any other PSI, i.e., patient specific instrument) may be associated with a virtual 3D model to fabricate it, as a function of a virtual 3D model of the bone, and/or of the implant that has been selected. More particularly, in the conception of the PSI, its virtual model may be applied to the virtual model of the bone and/or of the implant, emulating with precision the subsequent PSI T4 and bone relation. The PSI T4 may include a post T41 projecting from the tool body T2, and having at a free end thereof a guide T42 that will slide against the bone surface as the blade T1 resects the bone. The post T41 could be telescopic, and could be locked at a precise height. In femoral implant resection, a key resection is that of the anterior plane or posterior plane (once the other of the posterior plane or anterior plane is resected). Indeed, it is important for the thickness of the femur after the resection leading to the posterior plane and anterior plane to be within a limited range, to ensure a proper fit of the implant onto the distal femur. Accordingly, the PSI T4 may be used on the tool T manipulated by the robot arm 40 to resect a second of the two vertical planes of the femur (i.e., posterior plane and anterior plane). The use of PSI T4 by the robot arm 40 limits any deflection by the PSI T4 defining a rigid guide.

The present disclosure refers to the system 10 as performing continuous tracking. This means that the tracking may be performed continuously during discrete time periods of a surgical procedure. Continuous tracking may entail pauses, for example when the bone is not being altered. However, when tracking is required, the system 10 may provide a continuous tracking output, with any disruption in the tracking output triggering an alarm or message to an operator. The frequency of tracking may vary.

Thus, the system of the present disclosure can generally be described as bring a system for performing bone resection on a bone in robotized computer-assisted surgery, comprising: a processing unit; and a non-transitory computer-readable memory communicatively coupled to the processing unit and comprising computer-readable program instructions executable by the processing unit for: tracking a robot arm and a bone in a common referential system; obtaining a virtual model of a cutting block to be used on the bone, the virtual model including slots corresponding to at least one cutting plane of the cutting block; and controlling the robot arm having the cutting block supported at an end thereof to place the cutting block in a desired location on the bone, using the model of the cutting block and the tracking.

1. A system for performing bone resection on a bone in robotized computer-assisted surgery, comprising:
a processing unit; and
a non-transitory computer-readable memory communicatively coupled to the processing unit and comprising computer-readable program instructions executable by the processing unit for:
tracking a robot arm and a bone in a common referential system;
obtaining a virtual model of a cutting block to be used on the bone, the virtual model including slots corresponding to at least one cutting plane of the cutting block; and
controlling the robot arm having the cutting block supported at an end thereof to place the cutting block in a desired location on the bone, using the model of the cutting block and the tracking.

2. The system according to claim 1, wherein the computer-readable program instructions are executable by the processing unit for recording points on the bone in the common referential system, using the robot arm.

3. The system according to claim 1, wherein the bone is a femur and wherein the computer-readable program instructions are executable by the processing unit for controlling the robot arm in the desired location against a distal cut of the femur.

4. The system according to claim 3, wherein the computer-readable program instructions are executable by the processing unit for obtaining the virtual model of the cutting block with the at least one cutting plane being an anterior cut plane and a posterior cut plane.

5. The system according to claim 4, wherein the computer-readable program instructions are executable by the processing unit for obtaining the virtual model of the cutting block with the cutting planes including an anterior chamfer cut plane and a posterior chamfer cut plane.

6. The system according to claim 2, wherein the computer-readable program instructions are executable by the processing unit for obtaining a virtual model of a distal cutting block to be used on the femur, the virtual model including at least a cutting plane corresponding to a distal cut of the femur.

7. The system according to claim 6, wherein the computer-readable program instructions are executable by the processing unit for controlling the robot arm having the distal cutting block supported at an end thereof to place the distal cutting block in a desired location on the femur, using the model of the distal cutting block and the tracking.

8. The system according to claim 2, wherein the computer-readable program instructions are executable by the processing unit for controlling the robot arm to perform a distal cut.

9. The system according to claim 2, wherein the computer-readable program instructions are executable by the processing unit for controlling the robot arm to drill a peg hole in the femur.

10. The system according to claim 1, wherein the computer-readable program instructions are executable by the processing unit for controlling the robot arm having a cutting tool supported at the end thereof to position itself relative to the cutting block.

11. The system according to claim 1, wherein the computer-readable program instructions are executable by the processing unit for obtaining the virtual model of the cutting block from a manufacturer file.

12. The system according to claim 1, wherein the computer-readable program instructions are executable by the processing unit for:
tracking a tool relative to the bone and to the cutting block;
merging virtual models of the tool and the bone to the tool and the bone in the tracking;
calculating a location of a working end of the tool relative to the bone using the tracking, in a concealed condition of the working end of the tool relative to the bone while in the cutting block; and
outputting the location of the working end of the tool relative to the bone.

13. The system according to claim 12, wherein the computer-readable program instructions are executable by the processing unit for indicating a proximity of the working end with a boundary of the bone.

14. The system according to claim 12, wherein the computer-readable program instructions are executable by the processing unit for stopping an actuation of the tool when the working end is at the boundary of the bone.

15. The system according to claim 12, wherein the computer-readable program instructions are executable by the processing unit for imaging and displaying the concealed working end of the tool relative to the bone.

16. The system according to claim 1, including the robot arm.

17. The system according to claim 1, including the cutting block.

18. The system according to claim 17, wherein the cutting block has at least four cut slots.

19. The system according to claim 1, further including a saw configured for performing planar cuts of the bone as used with the cutting block.

20. The system according to claim 1, further including a drill configured for drilling holes in the bone to secure the cutting block to the bone.
